(19) United States
(12) Patent Application Publication
(10) Pub. No.: US 2024/0158731 A1
(43) Pub. Date: May 16, 2024

(54) ARRAY OF PLASMA SOURCES AND METHOD FOR TREATING CELL MATERIAL

(71) Applicant: ECERAMIX GMBH, Ilmenau (DE)

(72) Inventors: Nam GUTZEIT, Ilmenau (DE); Michael FISCHER, Uhlstädt-Kirchhasel (DE)

(21) Appl. No.: 18/550,128

(22) PCT Filed: Mar. 11, 2022
(86) PCT No.: PCT/EP2022/056303

§ 371 Date: Sep. 11, 2023

(30) Foreign Application Priority Data
Mar. 12, 2022 (DE) 10 2021 106 047.8

(51) Int. Cl. C12M 1/42; C12M 1/00; C12M 1/12; C12M 1/32

(52) U.S. Cl. CPC C12M 35/08; C12M 23/12; C12M 23/34; C12M 23/38; C12M 25/04

(57) ABSTRACT

The invention relates to an array (1) of plasma sources (3), comprising a carrier (2) on which a plurality of plasma sources (3) is arranged, wherein each plasma source (3) comprises an LTCC substrate or another ceramic substrate or a glass substrate or a silicon based substrate or a silicone based substrate or a composite substrate of glass and/or ceramics and/or plastics and/or silicon and/or silicone, wherein the carrier (2) is configured as a lid or part of a lid designed to be put on an array (6) of cell culture receptacles (9), such that an individual plasma source (3) is assigned to each cell culture receptacle (9), wherein each plasma source (3) is connected to at least one connection line (4). Moreover, the invention relates to a method for treating cell material (10) by the array (1) of plasma sources (3).

The invention relates to an array of plasma sources and to a method for treating cell material by means of the array of plasma sources.

Controlling germs in general by means of cold plasma (physical disinfection) is known in the art. Typically, a plasma jet process with pressurized gas is applied herein. However, the effect of the plasma generated this way is too intense for permanently treating cell material.

Moreover, in the field of molecular biology, controlling germs, in particular bacteria, in multiwell plates by antibiotics is known in the art. This is linked with the risk of antibiotic resistance and unwanted interference with the cell physiology by chemical substances (e.g. antibiotics) and also causes considerable effort for adding the medicaments/antibiotics.

The object of the invention is to provide an improved way for treating cell material.

According to the invention, the object is achieved by an array of plasma sources according to claim 1 and by a method for treating cell material according to claim 13.

Advantageous embodiments of the invention are subject of the dependent claims.

According to the invention, an array of plasma sources comprises a carrier on which a plurality of plasma sources is arranged, wherein each plasma source comprises a substrate, e.g. an LTCC substrate or another ceramic substrate or a glass substrate or a plastic based substrate or a silicon based substrate or a silicone based substrate or a composite substrate of glass and/or ceramics and/or plastics and/or silicon and/or silicone, wherein the carrier is configured as a lid or part of a lid designed to be put on a an array of cell culture receptacles, such that an individual plasma source is assigned to each cell culture receptacle, wherein each plasma source is connected to at least one connection line which may be led out of the carrier and be connectable or connected with an external control device. In an embodiment, each plasma source may be connected to at least two connection lines. In an embodiment, a ground potential, to which multiple plasma sources or all plasma sources may be connected, may be led in the carrier or in the array of cell culture receptacles.

This facilitates simultaneous plasma treatment of a plurality of cell material samples. The main effects of the plasma generated by the plasma sources are: emission of UV radiation, generation of reactive oxygen and nitrogen radicals and generation of ozone.

In an embodiment, the substrate of the plasma sources has conductor paths comprising gold or consisting of gold. In the alternative, other conductive materials may be used.

In an embodiment, each plasma source has an individual substrate. In the alternative, multiple plasma sources or all plasma sources may have a common substrate.

In an embodiment, the connection lines are individually assigned to the respective plasma source. In the alternative, multiple plasma sources have common connection lines.

In an embodiment, the plasma sources, individually or combined in groups, are controllable by an external control device using high voltage which may be varied with regard to amplitude, pulse form and pulse length.

In an embodiment, the carrier comprises a housing element having protrusions on which one of the plasma sources is arranged in each case.

In an embodiment, the housing element is configured to seal against an upper edge of the cell culture receptacles of the array.

According to an aspect of the present invention, a method for treating cell material, e.g. a 3D cell culture, by means of the above described array of plasma sources is provided, wherein an array of cell culture receptacles is provided, wherein cell material is inserted into at least one of the cell culture receptacles, in particular into multiple or all cell culture receptacles, wherein the array of plasma sources is put on the array of cell culture receptacles, wherein a cold, atmospheric plasma is generated by controlling at least one of the plasma sources, in particular multiple plasma sources or all plasma sources, of the array of plasma sources, and the cell material is treated with the plasma.

In an embodiment, the array of plasma sources is put on the array of cell culture receptacles prior to insertion of cell material, and a plasma treatment of the array of cell culture receptacles is carried out.

The present invention facilitates control of germs, in particular bacteria, in arrays of cell culture receptacles, e.g. multiwell plates. To date, antibiotics have been used for this purpose. On the contrary, the present invention allows for doing without antibiotics as the treatment is based on a purely physical mode of action. This also allows for controlling multiresistant germs. Moreover, due to the omission of antibiotics, a simplified handling of the arrays of cell culture receptacles results. Individual or group wise controllability of the individual plasma sources allows for finely dosing the plasma intensity for each position or for groups of positions of the array of cell culture receptacles. Commercially available plasma sources are too large for the application mentioned and/or their plasma intensity is too strong and/or cannot be dosed sufficiently, in particular since their intensity cannot be controlled and/or since the gas pressure required and/or the gas flow rate is too high.

The present invention allows for simplifying and improving experiments and analyses with cell cultures in the field of molecular biology, it may improve cell physiology and control bacteria, viruses or fungi in the cell culture. Moreover, the present invention may be used for disinfection in general. The disinfection may in particular be controlled by an adaptable plasma treatment for each individual cell culture receptacle, even over prolonged time periods.

In the following, embodiments of the invention will be described in more detail with reference to drawings.

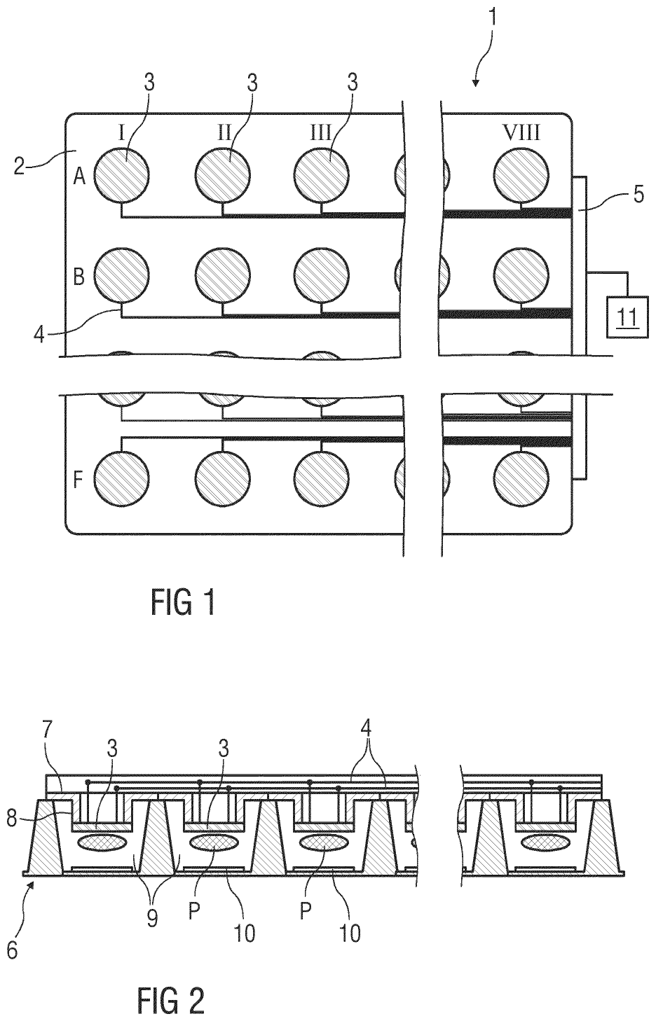

FIG. 1 is a schematic view of an array of plasma sources, and

FIG. 2 a schematic sectional view of the array of plasma sources.

Corresponding parts are provided with the same reference signs in both figures. FIG. 1 is a schematic view of an array 1 of plasma sources 3, comprising a carrier 2, wherein a plurality of plasma sources 3 is arranged on the carrier 2.

FIG. 2 is a schematic sectional view of the array 1 of plasma sources 3. For example, each of the plasma sources 3 is produced using the LTCC technology, e.g. as described in the publication M. Fischer et al., “LTCC-based micro plasma source for the selective treatment of cell cultures,” 2017 21st European Microelectronics and Packaging Conference (EMPC) & Exhibition, Warsaw, Poland, 2017, pp. 1-5, doi: 10.23919/EMPC.2017.8346855, which is herewith incorporated by reference into the present patent application in its entirety. The plasma sources 3 are in particular configured as multilayer ceramic plasma chips. Producing plasma sources 3 from multilayer ceramics allows for miniaturizing and, at the same time, for integrating contacts and electronics required for the plasma sources 3. This miniaturization is facilitated in particular by combining screen printing processes and laser structuring processes using ultra short pulse lasers. Other than with copper or silver as conductor path material which exhibits biocide properties, using gold or platinum conductor paths in LTCC substrates prevents adverse effects on cell material 10 to be treated.

Each plasma source 3 may be arranged on an individual substrate, e.g. an LTCC substrate, respectively. In the alternative, multiple or all plasma sources 3 may be arranged on a common substrate, e.g. an LTCC substrate.

In alternative embodiments, each plasma source 3 may be formed on an individual or common substrate of glass or another ceramic or a plastic based substrate or a silicon based substrate or a silicone based substrate or a composite substrate of glass and/or ceramics and/or plastics and/or silicon and/or silicone.

The carrier 2 may be configured as a lid designed to be put on an array 6 of cell culture receptacles 9, in particular a multiwell plate, a deep well plate or another arrangement of interconnected, in particular periodically arranged cell culture receptacles 9. The plasma sources 3 may be arranged in rows I to VIII and columns A to F as shown in FIG. 1. The quantity of plasma sources 3 shown is chosen as an example only though. The skilled person readily understands that a greater or smaller number of rows and/or columns may likewise be provided, in particular depending on the setup of the array 6 of cell culture receptacles 9.

Each of the plasma sources 3 may be connected to at least one connection line 4 respectively, e.g. two connection lines 4, which are assigned to the respective plasma source 3 such that the plasma source 3 may be controlled individually. Likewise, multiple plasma sources 3 may have common connection lines 4 and may thus be controlled as a group. Moreover, a ground potential, to which multiple plasma sources 9 or all plasma sources 9 may be connected, may be led in the carrier 2 and/or in the array 6 of cell culture receptacles 9. The connection lines 4 are for example led out of the carrier 2 via a connection piece 5 for contacting, e.g. by means of a plug connector. For example, the plasma sources 3 are simultaneously, e.g. periodically in time, controlled by an external control device 11, in particular individually or combined in groups. In particular, a high voltage is varied herein with regard to amplitude, pulse form and pulse length for controlling the plasma sources 3. This allows for a variation of the plasma treatment and thus for extensive treatment variations with individually adaptable treatment parameters. For example, alternating high voltage with frequencies in the MHz range, which may be pulsed in the kHz range, may be applied for the control.

The carrier 2 may comprise a housing element 7 which may have protrusions 8 on which one of the plasma sources 3 is arranged in each case, which therefore protrude into one respective cell culture receptacles 9 of the array 6 in order to bring a plasma P generated by the plasma source 3 as close as possible to a cell material 10 which may be located on the bottom of the cell culture receptacle 9. The housing element 7 may be configured to seal against an upper edge of the cell culture receptacles 9 of the array 6 so that the cell culture receptacles 9 of the array 6 are segregated from each other with the array 1 of plasma sources 3 put on.

In particular, a cold, atmospheric plasma may be generated and used for treating cell material 10, e.g. a 3D cell culture, by means of the array 1 of plasma sources 3 according to the invention. Possible fields of application of this technology are for example molecular biology, active substance screening and high throughput screening.

In addition, a plasma treatment of the unused, fresh array 6 of cell culture receptacles 9, e.g. a multiwell plate, may be carried out by means of the array 1 of plasma sources 3 according to the invention prior to introduction of biological material. This allows for continuously keeping the array 6 of cell culture receptacles 9 sterile as well as for a targeted influence or manipulation of the surface characteristics, e.g. an activation or a change of state, of the base material of the array 6 of cell culture receptacles 9 or of a coating which is optionally applied thereon.

The array 1 of plasma sources 3 may be produced adapted to different geometries of arrays 6 of cell culture receptacles 9.

### LIST OF REFERENCES

1 array of plasma sources
2 carrier
3 plasma source
4 connection line
5 connection piece
6 array of cell culture receptacles
7 housing element
8 protrusions
9 cell culture receptacles
10 cell material
11 control device
P plasma

1. An array of plasma sources, comprising a carrier on which a plurality of plasma sources is arranged, wherein each plasma source comprises one of:
a ceramic substrate,
an LTCC substrate,
a glass substrate,
a plastic based substrate,
a silicon based substrate,
a silicone based substrate,
a composite substrate of at least one of glass, ceramics, plastics, silicon and silicone,
wherein the carrier is configured as a lid or part of a lid designed to be put on an array of cell culture receptacles, such that an individual plasma source is assigned to each cell culture receptacle, wherein each plasma source is connected to at least one connection line.

2. The array of plasma sources according to claim 1, wherein each plasma source is connected to at least two connection lines.

3. The array of plasma sources according to claim 1, wherein a ground potential, to which multiple plasma sources or all plasma sources are connected, is led in the carrier or in the array of cell culture receptacles.

4. The array of plasma sources according to claim 1, wherein the substrate of the plasma sources has conductor paths comprising gold.

5. The array of plasma sources according to claim 1, wherein each plasma source has an individual substrate.

6. The array of plasma sources according to claim 1, wherein multiple or all plasma sources have a common substrate.

7. The array of plasma sources according to claim 1, wherein the connection lines are individually assigned to the respective plasma source.

8. The array of plasma sources according to claim 1, wherein multiple plasma sources have common connection lines.

9. The array of plasma sources according to claim 1, wherein the plasma sources, individually or combined in groups, are controllable by an external control device with high voltage.

10. The array of plasma sources according to claim 9, wherein the control device is configured to vary the high voltage with regard to amplitude, pulse form and pulse length.

11. The array of plasma sources according to claim 1, wherein the carrier comprises a housing element having protrusions on which one of the plasma sources is arranged in each case.

12. The array of plasma sources according to claim 1, wherein the carrier comprises a housing element and the housing element is configured to seal against an upper edge of the cell culture receptacles of the array.

13. A method for treating cell material by the array of plasma sources according to claim 1, wherein an array of cell culture receptacles is provided, wherein cell material is inserted into at least one of the cell culture receptacles, wherein the array of plasma sources is put on the array of cell culture receptacles, wherein a cold, atmospheric plasma is generated by controlling at least one of the plasma sources of the array of plasma sources, and the cell material is treated with the plasma.

14. The method according to claim 13, wherein the cold, atmospheric plasma is generated by controlling multiple plasma sources of the array of plasma sources.

15. The method according to claim 13, wherein the array of plasma sources is put on the array of cell culture receptacles prior to insertion of cell material, and a plasma treatment of the array of cell culture receptacles is carried out.
